(19) United States
(12) Patent Application Publication
(10) Pub. No.: US 2012/0029320 A1
(43) Pub. Date: Feb. 2, 2012

(54) SYSTEMS AND METHODS FOR PROCESSING MULTIPLE PHYSIOLOGICAL SIGNALS

(75) Inventors: James N. Watson, Dunfermline (GB); Robert S. Stoughton, Boulder, CO (US); Paul Stanley Addison, Edinburgh (GB)

(73) Assignee: Nellcor Puritan Bennett LLC, Boulder, CO (US)

(21) Appl. No.: 13/169,629

(22) Filed: Jun. 27, 2011

Related U.S. Application Data
(60) Provisional application No. 61/369,452, filed on Jul. 30, 2010.

(51) Int. Cl. A61B 5/00

(52) U.S. Cl. 600/301

(57) ABSTRACT

Systems and methods are provided for patient monitors which apply different sets of signal processing operations to signals to identify multiple fiducials in physiological signals. PPG signals measured at two sensor sites may be processed with a first set of processing operations and analyzed to identify fiducials that allow the calculation of a diastolic DPTT. These PPG signals may then be processed with a different set of processing operations and the results analyzed to identify fiducials that allow the calculation of a systolic DPTT.

### CROSS-REFERENCE TO RELATED APPLICATIONS

This application claims priority to U.S. Provisional Patent Application No. 61/369,452, “SYSTEMS AND METHODS FOR PROCESSING MULTIPLE PHYSIOLOGICAL SIGNALS,” filed Jul. 30, 2010 and incorporated by reference in its entirety herein.

### SUMMARY

Continuous non-invasive blood pressure (CNIBP) monitoring systems allow a patient's blood pressure to be tracked continuously, unlike standard occlusion cuff techniques, and without the hazards of invasive arterial lines. Some such systems use multiple pulse oximetry type sensors located at multiple body sites on a patient to measure photoplethysmograph (PPG) signals. The resulting multiple PPG signals may be compared against each other to estimate the patient's blood pressure. When the locations of two sensors are at different distances from the heart or along different paths from the heart (e.g., at the finger and forehead), a differential pulse transit time (DPTT) may be determined. A DPTT may represent the difference in the arrival times of a portion of a cardiac wave between the two locations, and may be determined by comparing corresponding fiducial points in the two PPG signals (e.g., a maximum, minimum, or a notch). In some techniques, two DPTTs are determined in order to calculate multiple physiological parameters, such as systolic and diastolic blood pressure. These DPTTs may be determined during different phases of the PPG signal representing different physiological occurrences. For example, one DPTT may be determined when the cardiovascular system is in a systolic state and a second DPTT may be determined when the cardiovascular system is in a diastolic state.

The accuracy of a blood pressure calculation based on a DPTT determination may depend on the accuracy of the DPTT determination. For more accurate transit times to be measured, it is desirable for the fiducial points to be easily and unambiguously resolved. However, fiducial points may not always be confidently identified by examining a raw PPG signal. For example, the diastolic portion of a PPG signal often has a sharp trough whose minimum point serves as an easily identifiable fiducial point. However, the peaks of a PPG signal (corresponding to the systolic portion) are often rounded and exhibit wide“shoulders” that make it difficult to distinguish a unique maximum (particularly in signals measured at the finger and forehead). Further, the peaks of PPG signals often change maxima positions between cardiac cycles. To sharpen the peaks, existing systems may apply a single signal processing technique, such as differentiating the PPG signal. While differentiation may disambiguate the position of a peak point associated with the systolic period of the cardiac cycle, it can hinder finding a diastolic fiducial.

Systems and methods are provided herein for determining physiological information about a subject with a monitoring device. The device receives a plurality of physiological signals of a subject from one or more sensors and uses a processor to generate a first plurality of processed signals by applying a first set of processing operations to the plurality of physiological signals. The device also generates a second plurality of processed signals by applying a second set of processing operations to the plurality of physiological signals, with the second set of processing operations being different from the first set of processing operations. The device calculates a first parameter by comparing features of the first plurality of processed signals, and calculate a second parameter by comparing features of the second plurality of processed signals. The device then determines physiological information about the subject based at least in part on the first and second parameters.

In some embodiments, the plurality of physiological signals includes one or more photoplethysmograph signals. Multiple photoplethysmograph signals may be measured at multiple different body sites of the subject. The first set of processing operations may include taking one or more time derivatives and/or applying a low-pass filter prior to taking one or more time derivatives. In some embodiments, the first set of processing operations includes taking a single time derivative and the second set of processing operations includes taking two time derivatives.

In some embodiments, the device calculates the first parameter by comparing extrema between one or more of the first plurality of processed signals. The first parameter may be a differential pulse transit time and/or may be determined based at least in part on a linear combination of multiple fiducial points in the first plurality of processed signals. In some embodiments, the physiological information includes a blood pressure.

In certain embodiments, the systems and methods described herein are used in

CNIBP monitors which apply different sets of signal processing operations to signals to identify multiple fiducials. In some embodiments, PPG signals measured at two sensor sites may be processed with a first set of processing operations and compared to identify fiducials that allow the calculation of a diastolic DPTT. These PPG signals may then be processed with a different set of processing operations and the results compared to identify fiducials that allow the calculation of a systolic DPTT.

The methods and systems of the present disclosure will be illustrated with reference to the monitoring of a physiological signal (which may be a PPG signal); however, it will be understood that the disclosure is not limited to monitoring physiological signals and is usefully applied within a number of signal monitoring settings.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 illustrates a methodology using different signal processing operations that may be applied to a signal in accordance with an embodiment;

FIG. 2(a) shows an illustrative patient monitoring system in accordance with an embodiment;

FIG. 2(b) is a block diagram of the illustrative patient monitoring system of FIG. 2(a) coupled to a patient in accordance with an embodiment;

FIG. 3 is a block diagram of an illustrative signal processing system in accordance with an embodiment;

FIG. 4 is an illustrative signal which may be analyzed in accordance with an embodiment; and

FIG. 5 is a flow chart of an illustrative process for determining physiological information in accordance with an embodiment.

### DETAILED DESCRIPTION

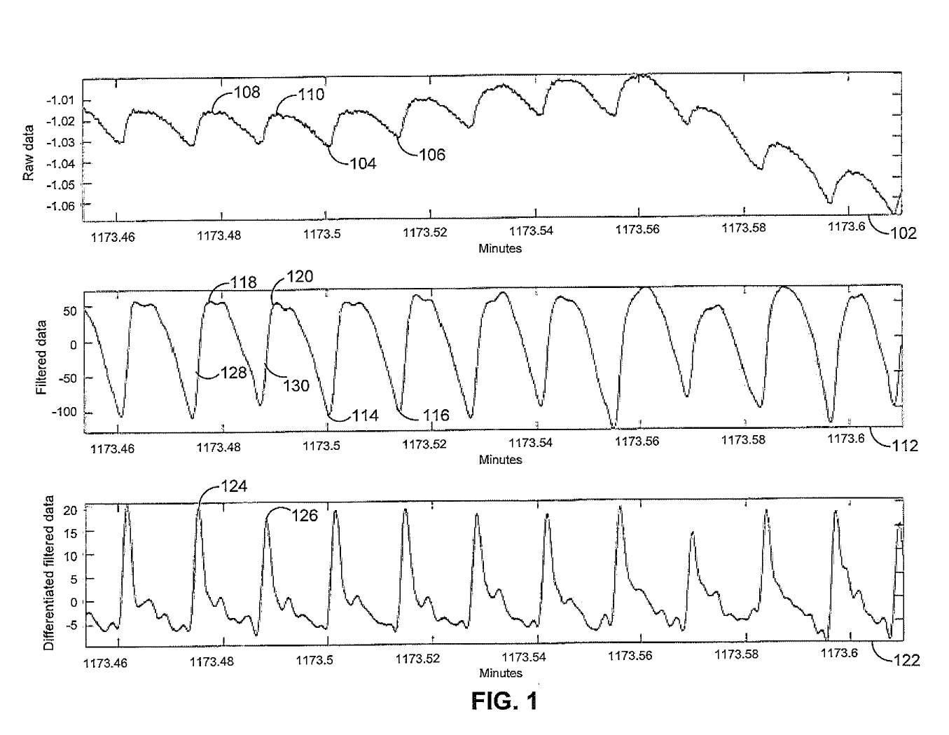

As described above, information about a system, such as the physiological system of human subject, may be determined by applying multiple signal processing techniques to a set of signals. FIG. 1 depicts an illustrative example of multiple signal processing techniques applied to a single signal in accordance with the systems and methods described herein. In particular, plot 102 of FIG. 1 depicts a raw photoplethysmograph (PPG) signal generated by a reflectance probe positioned on a subject's forehead for approximately ten seconds. Though the location of the troughs of this signal (e.g., troughs 104 and 106) may be more easily distinguished than the peaks (e.g., peaks 108 and 110), neither peaks nor troughs are particularly distinct.

Plot 112 of FIG. 1 depicts the raw PPG signal of plot 102 after the PPG signal has been filtered with a band-pass filter with a pass-band of approximately 0.5 Hz-7.5 Hz. This filtering has sharpened the troughs (e.g., troughs 114 and 116), allowing their locations to be more easily identified. Physiological parameter calculations based on the locations of these troughs as fiducial points, such as a diastolic blood pressure calculation, may be more accurate than calculations based on the troughs of the raw PPG signal of plot 102. However, the peaks of the filtered signal (e.g., peaks 118 and 120) may not exhibit a similar improvement in discernability over the peaks of the raw PPG signal of plot 102, and indeed may be more difficult to distinguish than their counterparts in the raw PPG signal of plot 102. These peaks may not readily provide accurate fiducial points to use in physiological parameter calculations. In calculations which require the identification of more than one fiducial point, a different approach may be desired.

Plot 122 of FIG. 1 depicts a time derivative of the filtered signal of plot 112. The locations of the peaks of plot 122 (e.g., peaks 124 and 126) are more easily distinguished than the peaks of plot 112. The peaks of plot 122 correspond to the points of maximum slope in plot 112 (i.e. the upstrokes of the PPG signal). For example, peak 124 corresponds to upstroke 128 of plot 112 and peak 126 corresponds to upstroke 130 of plot 112. The peaks of plot 122 may be used as fiducial points in a physiological parameter calculation, such as a systolic blood pressure calculation. The peaks of plot 122 may be used in combination with the fiducial points given by the troughs of plot 112 to determine a subject's systolic and diastolic blood pressure. In an embodiment, as discussed above, two different fiducial points, identified in signals measured at two different body sites of a subject, may allow two differential pulse transit times (DPTTs) to be calculated, which may then be used to determine the subject's systolic and diastolic blood pressure. Thus, different fiducial points may be identified from a physiological signal by applying different signal processing operations and locating fiducial points or features in the processed signals. The present disclosure relates to systems and methods for applying multiple processing operations to one or more physiological signals in order to identify multiple parameters based on features of the multiple processed physiological signals.

For illustrative purposes, the systems and techniques disclosed herein may be described in the context of continuous, non-invasive blood pressure monitoring (CNIBP) systems, oximetry systems, and other patient monitoring systems. However, the disclosed systems and methods may be suitable for any signal processing and monitoring application in which different fiducial points may be identified in multiple signals. In particular, the systems and methods described herein have application in any methodology that requires the identification of multiple fiducials from any periodic signal or any collection of signals.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot.

The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
I₀=intensity of light transmitted;
s=oxygen saturation;
β₀, βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.
1. The natural logarithm of Eq. 1 is taken (“log” will be used to represent the natural logarithm) for IR and Red to yield

log I=log I_{O}−(sβ_{O}+(1−s) βᵣ)l. (2)
2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix} } . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R , \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

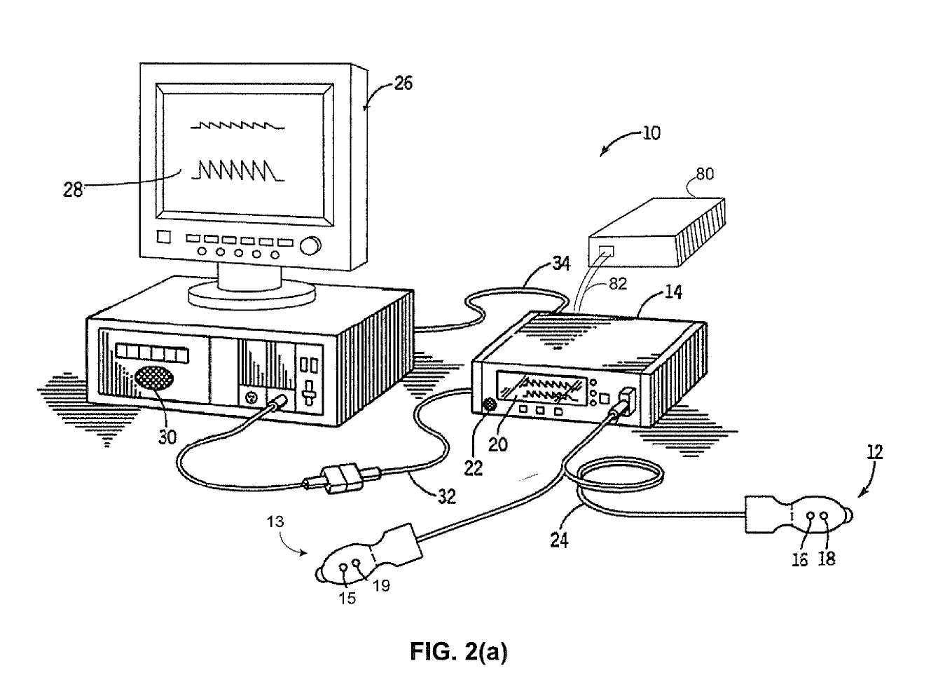

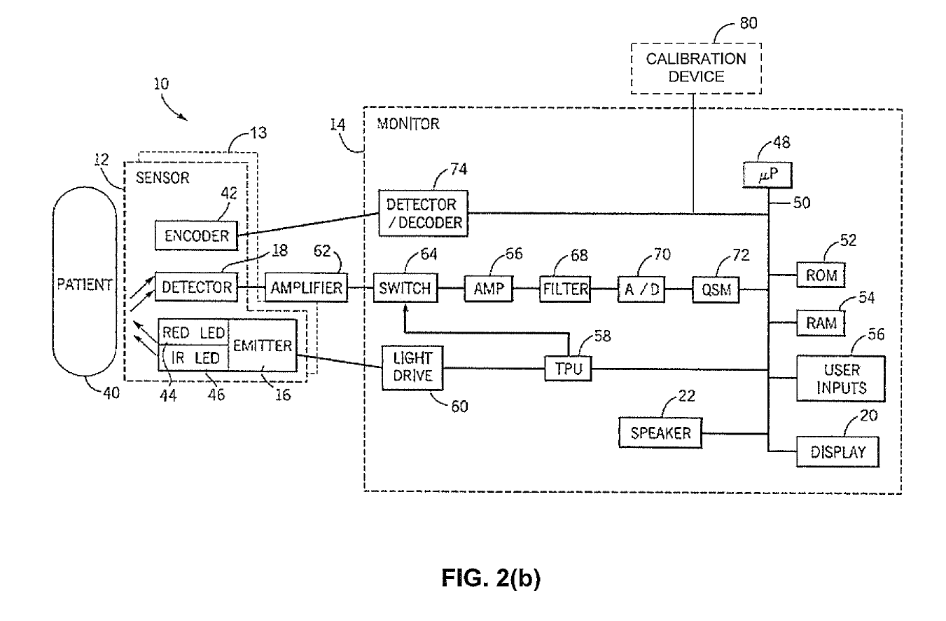

FIG. 2(a) is a perspective view of an embodiment of a patient monitoring system 10. System 10 may include sensor unit 12 and monitor 14. In an embodiment, sensor unit 12 may be part of a continuous, non-invasive blood pressure (CNIBP) monitoring system and/or an oximeter. Sensor unit 12 may include an emitter 16 for emitting light at one or more wavelengths into a patient's tissue. A detector 18 may also be provided in sensor 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units, such as sensor unit 13, which may take the form of any of the embodiments described herein with reference to sensor unit 12. For example, sensor unit 13 may include emitter 15 and detector 19. Sensor unit 13 may be the same type of sensor unit as sensor unit 12, or sensor unit 13 may be of a different sensor unit type than sensor unit 12. Sensor units 12 and 13 may be capable of being positioned at two different locations on a subject's body; for example, sensor unit 12 may be positioned on a patient's forehead, while sensor unit 13 may be positioned at a patient's fingertip. [0026] Sensor units 12 and 13 may each detect any signal that carries information about a patient's physiological state, such as an electrocardiograph signal, arterial line measurements, or the pulsatile force exerted on the walls of an artery using, for example, oscillometric methods with a piezoelectric transducer. According to another embodiment, system 10 may include a plurality of sensors forming a sensor array in lieu of either or both of sensor units 12 and 13. Each of the sensors of a sensor array may be a complementary metal oxide semiconductor (CMOS) sensor. Alternatively, each sensor of an array may be charged coupled device (CCD) sensor. In an embodiment, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more of sensor units 12 and 13 in accordance with the systems and techniques disclosed herein. It is understood that any number of sensors measuring any number of physiological signals may be used to determine physiological information in accordance with the techniques described herein.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as in a sensor designed to obtain pulse oximetry data from a patient's forehead.

In an embodiment, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., heart rate, blood pressure, blood oxygen saturation) based at least in part on data relating to light emission and detection received from one or more sensor units such as sensor units 12 and 13. In an alternative embodiment, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In an embodiment, the monitor 14 includes a blood pressure monitor. In alternative embodiments, the system 10 includes a stand-alone blood pressure monitor in communication with the monitor 14 via a cable or a wireless network link.

In an embodiment, sensor unit 12 may be communicatively coupled to monitor 14 via a cable 24. However, in other embodiments, a wireless transmission device (not shown) or the like may be used instead of or in addition to cable 24.

In the illustrated embodiment, system 10 includes a multi-parameter patient monitor 26. The monitor 26 may include a cathode ray tube display, a flat panel display (as shown) such as a liquid crystal display (LCD) or a plasma display, or may include any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by monitor 14 (referred to as an “SpO₂” measurement), pulse rate information from monitor 14 and blood pressure from monitor 14 on display 28. Multi-parameter patient monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

Calibration device 80, which may be powered by monitor 14 via a cable 82, a battery, or by a conventional power source such as a wall outlet, may include any suitable signal calibration device. Calibration device 80 may be communicatively coupled to monitor 14 via cable 82, and/or may communicate wirelessly (not shown). In other embodiments, calibration device 80 is completely integrated within monitor 14. For example, calibration device 80 may take the form of any invasive or non-invasive blood pressure monitoring or measuring system used to generate reference blood pressure measurements for use in calibrating a CNIBP monitoring technique as described herein. Such calibration devices may include, for example, an aneroid or mercury sphygmomanometer and occluding cuff, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference signal measurements obtained from some other source (e.g., an external invasive or non-invasive physiological measurement system).

Calibration device 80 may also access reference signal measurements stored in memory (e.g., RAM, ROM, or a storage device). For example, in some embodiments, calibration device 80 may access reference blood pressure measurements from a relational database stored within calibration device 80, monitor 14, or multi-parameter patient monitor 26. The reference blood pressure measurements generated or accessed by calibration device 80 may be updated in real-time, resulting in a continuous source of reference blood pressure measurements for use in continuous or periodic calibration. Alternatively, reference blood pressure measurements generated or accessed by calibration device 80 may be updated periodically, and calibration may be performed on the same periodic cycle or a different periodic cycle. Reference blood pressure measurements may be generated when recalibration is triggered.

FIG. 2(b) is a block diagram of a patient monitoring system, such as patient monitoring system 10 of FIG. 2(a), which may be coupled to a patient 40 in accordance with an embodiment. Certain illustrative components of sensor unit 12 and monitor 14 are illustrated in FIG. 2(b). Because sensor units 12 and 13 may include similar components and functionality, only sensor unit 12 will be discussed in detail for ease of illustration. It will be understood that any of the concepts, components, and operation discussed in connection with sensor unit 12 may be applied to sensor unit 13 as well (e.g., emitter 16 and detector 18 of sensor unit 12 may be similar to emitter 15 and detector 19 of sensor unit 13). It will be noted that patient monitoring system 10 may include one or more additional sensor units or probes, which may take the form of any of the embodiments described herein with reference to sensor units 12 and 13 (FIG. 2(a)). These additional sensor units included in system 10 may take the same form as sensor unit 12, or may take a different form. In an embodiment, multiple sensors (distributed in one or more sensor units) may be located at multiple different body sites on a patient.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In one embodiment, the Red wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a Red light while a second emits only an IR light. In another example, the wavelengths of light used are selected based on the specific location of the sensor.

It will be understood that, as used herein, the term “light” may refer to energy produced by radiation sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a pulse of a photoplethysmograph (PPG) signal to determine blood pressure. These equations may contain coefficients that depend upon a patient's physiological characteristics as stored in encoder 42. Encoder 42 may, for instance, be a coded resistor which stores values corresponding to the type of sensor unit 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 fills up. In one embodiment, there may be multiple separate parallel paths having components equivalent to amplifier 66, filter 68, and/or AID converter 70 for multiple light wavelengths or spectra received.

In an embodiment, microprocessor 48 may determine the patient's physiological parameters, such as SpO₂, pulse rate, and/or blood pressure, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals or otherwise identify noise components in order to prevent them from affecting measurements of physiological parameters derived from the sensor signals.

Pulse oximeters, in addition to providing other information, can be utilized for continuous non-invasive blood pressure monitoring. As described in Chen et al., U.S. Pat. No. 6,599,251, the entirety of which is incorporated herein by reference, PPG and other pulse signals obtained from multiple probes can be processed to calculate the blood pressure of a patient. In particular, blood pressure measurements may be derived based on a comparison of time differences between certain components of the pulse signals detected at each of the respective probes. As described in U.S. patent application Ser. No. 12/242,238, filed on Sep. 30, 2008 and entitled “Systems and Methods For Non-Invasive Blood Pressure Monitoring,” the entirety of which is incorporated herein by reference, blood pressure can also be derived by processing time delays detected within a single PPG or pulse signal obtained from a single pulse oximeter probe. In addition, as described in U.S. patent application Ser. No. 12/242,867, filed on Sep. 30, 2008 and entitled “Systems and Methods For Non-Invasive Continuous Blood Pressure Determination,” the entirety of which is incorporated herein by reference, blood pressure may also be obtained by calculating the area under certain portions of a pulse signal. Finally, as described in U.S. patent application Ser. No. 12/242,862, filed on Sep. 30, 2008 and entitled “Systems and Methods For Maintaining Blood Pressure Monitor Calibration,” the entirety of which is incorporated herein by reference, a blood pressure monitoring device may be recalibrated in response to arterial compliance changes.

As described above, some CNIBP monitoring techniques utilize two probes or sensors positioned at two different locations on a subject's body. The elapsed time, T, between the arrivals of corresponding points of a pulse signal at the two locations may then be determined using signals obtained by the two probes or sensors. The estimated blood pressure, p, may then be related to the elapsed time, T, by

p=a+b·ln(T) (14)

where a and b are constants that may be dependent upon the nature of the subject and the nature of the signal detecting devices. Other suitable equations using an elapsed time between corresponding points of a pulse signal may also be used to derive an estimated blood pressure measurement.

In an embodiment, Eq. 14 may include a non-linear function which is monotonically decreasing and concave upward in Tin a manner specified by the constant parameters (in addition to or instead of the expression of Eq. 14). Eq. 14 may be used to calculate an estimated blood pressure from the time difference T between corresponding points of a pulse signal received by two sensors or probes attached to two different locations of a subject.

In an embodiment, constants a and b in Eq. 14 above may be determined by performing a calibration. The calibration may involve taking a reference blood pressure reading to obtain a reference blood pressure P₀, measuring the elapsed time T₀ corresponding to the reference blood pressure, and then determining values for both of the constants a and b from the reference blood pressure and elapsed time measurement. Calibration may be performed at any suitable time (e.g., once initially after monitoring begins) or on any suitable schedule (e.g., a periodic or event-driven schedule).

In an embodiment, the calibration may include performing calculations mathematically equivalent to

$$\begin{matrix}a = {c}_{1} + \frac{{c}_{2} ( {P}_{0} - {c}_{1} )}{ln ( {T}_{0} ) + {c}_{2}} and & ( 15 ) \\ b = \frac{{P}_{0} - {c}_{1}}{ln ( {T}_{0} ) + {c}_{2}} & ( 16 )\end{matrix}$$

to obtain values for the constants a and b, where c₁ and c₂ are parameters that may be determined, for example, based on empirical data.

In an embodiment, the calibration may include performing calculations mathematically equivalent to

a=P₀−(c₃T₀+c₄)ln(T₀) (17)

and

b=c₃T₀+c₄ (18)

where a and b are first and second parameters and c₃ and c₄ are parameters that may be determined, for example, based on empirical data.

Parameters c₁, c₂, c₃, and c₄ may be predetermined constants empirically derived using experimental data from a number of different patients. A single reference blood pressure reading from a patient, including reference blood pressure P₀ and elapsed time T₀ from one or more signals corresponding to that reference blood pressure, may be combined with such inter-patient data to calculate the blood pressure of a patient. The values of P₀ and T₀ may be referred to herein as a calibration point. According to this example, a single calibration point may be used with the predetermined constant parameters to determine values of constants a and b for the patient (e.g., using Eqs. 15 and 16 or 17 and 18). The patient's blood pressure may then be calculated using Eq. 14. Recalibration may be performed by collecting a new calibration point and recalculating the constants a and b used in Eq. 14. Calibration and recalibration may be performed using calibration device 80 (FIG. 2(a)).

In an embodiment, multiple calibration points from a patient may be used to determine the relationship between the patient's blood pressure and one or more PPG signals. This relationship may be linear or non-linear and may be extrapolated and/or interpolated to define the relationship over the range of the collected recalibration data. For example, the multiple calibration points may be used to determine values for parameters c₁ and c₂ or c₃ and c₄ (described above). These determined values will be based on information about the patient (intra-patient data) instead of information that came from multiple patients (inter-patient data). As another example, the multiple calibration points may be used to determine values for parameters a and b (described above). Instead of calculating values of parameters a and b using a single calibration point and predetermined constants, values for parameters a and b may be empirically derived from the values of the multiple calibration points. As yet another example, the multiple calibration points may be used directly to determine the relationship between blood pressure and PPG signals. Instead of using a predefined relationship (e.g., the relationship defined by Eq. 14), a relationship may be directly determined from the calibration points.

Additional examples of continuous and non-invasive blood pressure monitoring techniques are described in Chen et al., U.S. Pat. No. 6,566,251, which is hereby incorporated by reference herein in its entirety. The technique described by Chen et al. may use two sensors (e.g., ultrasound or photoelectric pulse wave sensors) positioned at any two locations on a subject's body where pulse signals are readily detected. For example, sensors may be positioned on an earlobe and a finger, an earlobe and a toe, or a finger and a toe of a patient's body.

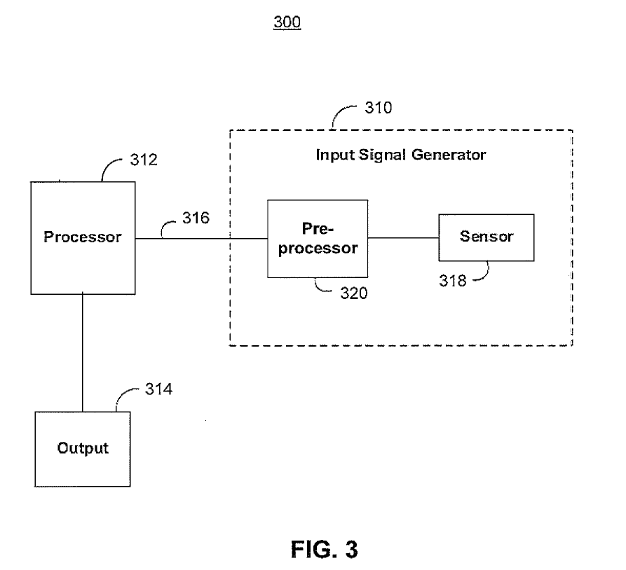

FIG. 3 is an illustrative signal processing system 300 in accordance with an embodiment that may implement the non-invasive blood pressure techniques described herein. In this embodiment, input signal generator 310 generates an input signal 316. As illustrated, input signal generator 310 may include pre-processor 320 coupled to sensor 318, which may provide input signal 316. In an embodiment, pre-processor 320 may be an oximeter and input signal 316 may be a PPG signal. In an embodiment, pre-processor 320 may be any suitable signal processing device and input signal 316 may include one or more PPG signals and one or more other physiological signals, such as an electrocardiogram (ECG) signal. It will be understood that input signal generator 310 may include any suitable signal source, signal generating data, signal generating equipment, or any combination thereof to produce signal 316. Signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing AID conversion. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In an embodiment, signal 316 may include PPG signals at one or more frequencies, such as a Red PPG signal and an IR PPG signal. In an embodiment, signal 316 may include signals measured at one or more sites on a patient's body, for example, a patient's finger, toe, ear, arm, or any other body site. In an embodiment, signal 316 may include multiple types of signals (e.g., one or more of an ECG signal, an EEG signal, an acoustic signal, an optical signal, a signal representing a blood pressure, and a signal representing a heart rate). Signal 316 may be any suitable biosignal or signals, such as, for example, electrocardiogram, electroencephalogram, electrogastrogram, electromyogram, heart rate signals, pathological sounds, ultrasound, or any other suitable biosignal. The systems and techniques described herein are also applicable to any dynamic signals, non-destructive testing signals, condition monitoring signals, fluid signals, geophysical signals, astronomical signals, electrical signals, financial signals including financial indices, sound and speech signals, chemical signals, meteorological signals including climate signals, any other suitable signal, and/or any combination thereof.

In an embodiment, signal 316 may be coupled to processor 312. Processor 312 may be any suitable software, firmware, hardware, or combination thereof for processing signal 316. For example, processor 312 may include one or more hardware processors (e.g., integrated circuits), one or more software modules, computer-readable media such as memory, firmware, or any combination thereof. Processor 312 may, for example, be a computer or may be one or more chips (i.e., integrated circuits). Processor 312 may, for example, be configured of analog electronic components. Processor 312 may perform the calculations associated with the information determination techniques of the present disclosure as well as the calculations associated with any calibration of processing system 300 or other auxiliary functions. For example, processor 312 may locate one or more fiducial points in one or more signals, determine one or more DPTTs, and compute one or more of a systolic blood pressure, a diastolic blood pressure and a mean arterial pressure. Processor 312 may perform any suitable signal processing of signal 316 to filter signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the patient. Additional input signals may be used by processor 312 in any of the calculations or operations it performs in accordance with processing system 300.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 312 to, for example, store data corresponding to blood pressure monitoring, including current blood pressure calibration values, blood pressure monitoring calibration thresholds, and patient blood pressure history. In an embodiment, processor 312 may store physiological measurements or previously received data from signal 316 in a memory device for later retrieval. In an embodiment, processor 312 may store calculated values, such as a systolic blood pressure, a diastolic blood pressure, a blood oxygen saturation, a differential pulse transit time, a fiducial point location or characteristic, or any other calculated values, in a memory device for later retrieval.

Processor 312 may be coupled to a calibration device. This coupling may take any of the forms described above with reference to calibration device 80 within system 10. For example, the calibration device may be a stand-alone device that may be in wireless communication with processor 312, or may be completely integrated with processor 312. [0061] Processor 312 may be coupled to a calibration device that may generate, or receive as input, reference measurements for use in calibration calculations. This coupling may occur through a recalibration signal transmitted via a wired or wireless communications path. In an embodiment, processor 312 is capable of transmitting a command to calibration device 80 to initiate a recalibration procedure.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g, hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as parts of sensor units 12 and 13 (FIGS. 1 and 2) and monitor 14 (FIGS. 1 and 2) and processor 312 may be implemented as part of monitor 14 (FIGS. 1 and 2). In sonic embodiments, portions of system 300 may be configured to be portable. For example, all or part of system 300 may be embedded in a small, compact object carried with or attached to the patient (e.g., a watch, other piece of jewelry, or a cellular telephone). In such embodiments, a wireless transceiver (not shown) may also be included in system 300 to enable wireless communication with other components of system 10 (FIGS. 1 and 2). As such, system 10 (FIGS. 1 and 2) may be part of a fully portable and continuous patient monitoring solution. In such embodiments, a wireless transceiver (not shown) may also be included in system 300 to enable wireless communication with other components of system 10. For example, pre-processor 320 may output signal 316 over BLUETOOTH, 802.11, WiFi, WiMax, cable, satellite, Infrared, or any other suitable transmission scheme. In an embodiment, a wireless transmission scheme may be used between any communicating components of system 300.

Pre-processor 320 or processor 312 may determine the locations of pulses within a periodic signal 316 (e.g., a PPG signal) using a pulse detection technique. For ease of illustration, the following pulse detection techniques will be described as performed by processor 312, but any suitable processing device (e.g., pre-processor 320) may be used to implement any of the techniques described herein.

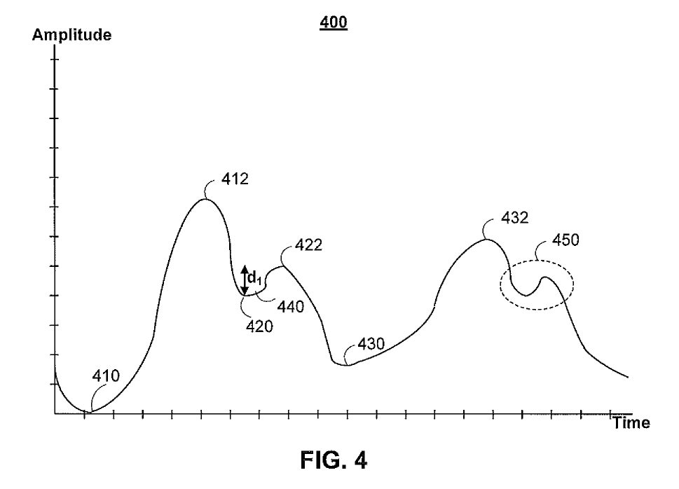

An illustrative PPG signal 400 is depicted in FIG. 4. Processor 312 may receive PPG signal 400, and may identify local minimum point 410, local maximum point 412, local minimum point 420, and local maximum point 422 in the PPG signal 400. Processor 312 may pair each local minimum point with an adjacent maximum point. For example, processor 312 may pair points 410 and 412 to identify one segment, points 412 and 420 to identify a second segment, points 420 and 422 to identify a third segment and points 422 and 430 to identify a fourth segment. The slope of each segment may be measured to determine whether the segment corresponds to an upstroke portion of the pulse (e.g., a positive slope) or a downstroke portion of the pulse (e.g., a negative slope) portion of the pulse. A pulse may be defined as a combination of at least one upstroke and one downstroke. For example, the segment identified by points 410 and 412 and the segment identified by points 412 and 420 may define a pulse.

According to an embodiment, PPG signal 400 may include a dichrotic notch 450 or other notches (not shown) in different sections of the pulse (e.g., at the beginning (referred to as an ankle notch), in the middle (referred to as a dichrotic notch), or near the top (referred to as a shoulder notch)). Processor 312 may identify notches and either utilize or ignore them when detecting the pulse locations. In some embodiments, processor 312 may compute the second derivative of the PPG signal to find the local minima and maxima points and may use this information to determine a location of, for example, a dichrotic notch. Additionally, processor 312 may interpolate between points in signal 316 or between points in a processed signal using any interpolation technique (e.g., zero-order hold, linear interpolation, and/or higher-order interpolation techniques). Some pulse detection techniques that may be performed by processor 312 are described in more detail in co-pending, commonly assigned U.S. patent application Ser. No. 12/242,908, filed Sep. 30, 2008 and entitled “SYSTEMS AND METHODS FOR DETECTING PULSES IN A PPG SIGNAL,” which is incorporated by reference herein in its entirety.

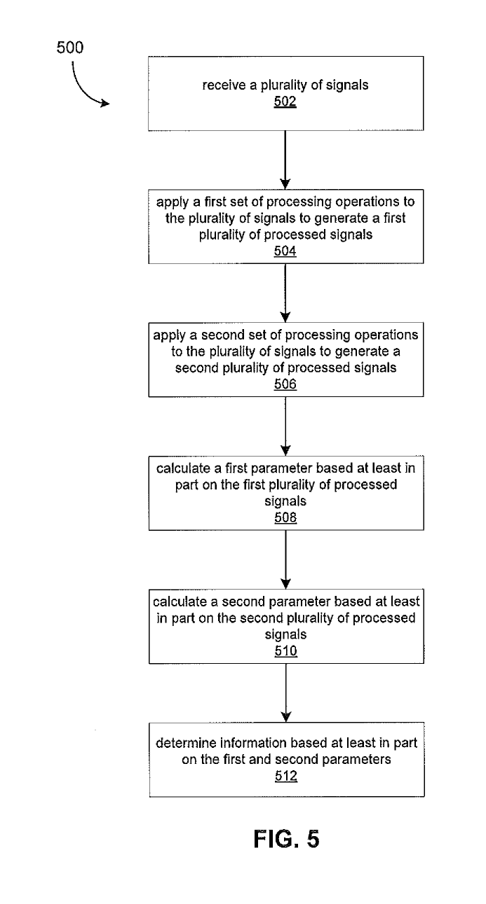

FIG. 5 is a flow diagram 500 of illustrative steps involved in determining information from monitored signals in accordance with an embodiment. The steps of flow diagram 500 may be performed by processor 312 (FIG. 3), or may be performed by any suitable processing device communicatively coupled to monitor 14 (FIGS. 1 and 2). The steps of flow diagram 500 may be performed by a digital processing device, or implemented in analog hardware. In an embodiment, the steps of flow diagram 500 may be performed by a continuous, non-invasive blood pressure (CNIBP) monitoring system. It will be noted that the steps of flow diagram 500 may be performed in any suitable order, and one or more steps may be omitted entirely according to the context and application.

At step 502, a plurality of signals may be received. A signal (e.g., a PPG signal) may be received from any suitable source (e.g., patient 40 of FIG. 2(b)) using any suitable technique. A received signal may be generated by sensor unit 12 and/or sensor unit 13 (FIG. 2(a)), which may each include any of the physiological sensors described herein, or any other sensor. A received signal may be signal 316, which may be generated by a pre-processor 320 coupled between processor 312 and sensor 318 (FIG. 3). A received signal may include multiple signals, for example, in the form of a multi-dimensional vector signal or a frequency- or time-multiplexed signal. In an embodiment, the plurality of signals received at step 502 may include two or more PPG signals, which may be measured at two or more respective different body sites of a subject.

The plurality of signals received at step 502 may include first and second physiological signals received as input signal 316 (FIG. 3). In an embodiment, a first signal may be a Red PPG signal, and a second signal may be an IR PPG signal. In an embodiment, first and second signals may be different types of signals (e.g., a PPG signal and an ECG signal). In an embodiment, first and second signals may be obtained by first and second sensors located at approximately the same body site of a subject. In an embodiment, first and second signals may be obtained by first and second sensors located at different body sites of a subject. For example, first and second signals included in the plurality of signals may be electronic signals from pulse oximetry sensors located at two different body sites of a subject.

In an embodiment, more than two signals may be received at step 502. For example, PPG signals at three or more frequencies may be obtained at step 502, or PPG signals from three or more body sites, or any set of three or more signals (such as two PPG signals and an ECG signal). It will be noted that the steps of flow diagram 500 may be applied to any number of received signals in accordance with the techniques described herein.

At step 504, one or more of the plurality of signals received at step 502 may be processed with a first set of processing operations This processing may result in a first plurality of processed signals. The processing may occur in conjunction with the receiving at step 502, or after the signals are received at step 502. A processing operation may be performed by any suitable processing device, such as processor 312 (FIG. 3) and/or microprocessor 48 (FIG. 2(b)), each of which may be a general-purpose computing device or a specialized processor. A processing operation may be performed by a separate, dedicated device, or by a series of devices (e.g., an analog filter and a programmed microprocessor).

Processor 312 (FIG. 3) may transform the original and/or transformed signals into any suitable domain. In an embodiment, the processing at step 504 may include transforming a signal into another domain, for example, a Fourier, wavelet, spectral, scale, time, time-spectral, time-scale domain, or any transform space. A transformation may include a continuous wavelet transformation as described, for example, in Paul S. Addison, The Illustrated Wavelet Transform Handbook (Taylor & Francis Group 2002), which is hereby incorporated by reference herein in its entirety.

The processing at step 504 may include filtering a signal 316 (FIG. 3) or mathematically manipulating one or multiple signals. For example, a processed signal may be a ratio of two signals. A processed signal may be based at least in part on past values of a signal, such as signal 316 (FIG. 3), which may be retrieved by processor 312 (FIG. 3) from a memory such as a buffer memory or RAM 54 (FIG. 2(b)). Many examples of processing operations are discussed in detail herein, but it will be understood that the techniques of the present disclosure are not limited to these examples.

In an embodiment, the first set of processing operations of step 504 may include any one or more of the following: compressing, multiplexing, modulating, up-sampling, down-sampling, smoothing, taking a median or other statistic of the received signal, removing erroneous regions of the received signal, or any combination thereof. In an embodiment, a normalization step may be performed which divides the magnitude of a signal received at step 502 by a value. This value may be based on at least one of the maximum of the received signal, the minimum of the received signal and the mean of the received signal. In an embodiment, a signal received at step 502 may be normalized by dividing the signal by a DC component. In an embodiment, a signal received at step 502 may be normalized by dividing the signal by the standard deviation of the signal computed over a time window. In an embodiment, the first set of processing operations at step 504 may include one or more mathematical manipulations. Mathematical manipulations may include any linear or non-linear combination or signals or portions of signals, and may be performed in any suitable domain (e.g., time, frequency and wavelet domains).

In an embodiment, the first set of processing operations at step 504 may include one or more time derivatives. A time derivative may be calculated by input signal generator 310 (FIG. 3) (alone or in conjunction with additional pre-processing steps), or may be calculated by processor 312 (FIG. 3). In an embodiment, a time derivative may be calculated by any of a number of derivative/gradient determination and approximation techniques, including those suitable for sampled data (e.g., forward difference, backward difference, central difference, higher-order methods, and any automated numerical or symbolic differentiation method).

In an embodiment, the first set of processing operations at step 504 may include filtering using any suitable filtering technique. For example, a signal received at sensor unit 12 (FIGS. 1 and 2) may be filtered at step 504 by low pass filter 68 (FIG. 2(b)) prior to undergoing additional processing at microprocessor 48 (FIG. 2(b)) within patient monitoring system 10 (FIGS. 1 and 2). Low pass filter 68 (FIG. 2(b)) may selectively remove frequencies that may later be ignored by further processing or analysis steps, which may advantageously reduce computational time and memory requirements. In an embodiment, one or more signals received at step 502 may be high or band pass filtered at step 504 to remove low frequencies. Such a filter may be, for example, a derivative filter. Taking a derivative of a signal may selectively emphasize the signal's high frequency components. In an embodiment, one or more signals received at step 502 may be filtered at step 504 to remove a DC component. In an embodiment, a PPG signal may be band-pass filtered at step 504 to pass frequencies in the approximate range 0.5-3 Hz. In an embodiment, a PPG signal may be band-pass filtered at step 504 to pass frequencies in the approximate range 0.5-7.5 Hz. In an embodiment, the cutoff frequencies of a filter may be chosen based on the frequency response of the hardware platform underlying patient monitoring system 10 (FIGS. 1 and 2). In an embodiment, a windowing operation may be performed at step 504 to suppress or amplify one or more portions of a signal received at step 502.

Different processing operations may be applied to any one or more of the first and second signals received at step 502 and/or any components of a multi-component signal. For example, different operations may be applied to a signal taken from a first body site and a signal taken from a second body site. In an embodiment, a first received signal may be passed through a high-pass filter and a second received signal may not be passed through a high-pass filter.

Any of the operations described herein may be applied to a portion or portions of a received signal. An operation may be broken into one or more stages performed by one or more devices within signal processing system 300 of FIG. 3 (which may itself be a part of patient monitoring system 10 of FIGS. 1 and 2). For example, a filtering technique may be applied by input signal generator 310 (FIG. 3) prior to passing the resulting input signal 316 (FIG. 3) to processor 312 (FIG, 3), where it may undergo a transformation and/or the calculation of a time derivative. Embodiments of the steps of flow diagram 500 include any of the operations described herein performed in any suitable order.

At step 506, a second plurality of processed signals may be generated by applying a second set of processing operations to one or more of the plurality of physiological signals received at step 502. The second set of processing operations may be performed in accordance with any one or more of the processing operations described herein, including any combination of any of the processing operations described above with reference to the first set of processing operations in step 504. In an embodiment, the second set of processing operations performed at step 506 is different from the first set of processing operations performed at step 504.

For example, as discussed above with reference to FIG. 1, the first set of processing operations may include applying a filtering operation, and the second set of processing operations may include applying the same filtering operation and then taking a time derivative. In an embodiment, the first set of processing operations may include a single time derivative and the second set of processing operations may include two time derivatives. In an embodiment, one or more of the first and second sets of processing operations may include taking N time derivatives, where N is a number greater than two. In an embodiment, a first set of processing operations may include taking M time derivatives and a second set of processing operations may include taking N time derivatives, where M and N are different.

At step 508, a first parameter may be calculated based at least in part on the first plurality of processed signals. In an embodiment, a first parameter may be calculated at step 508 by comparing features of two or more of the first plurality of processed signals. A feature of a signal or processed signal may be any characterization of that signal, including, for example, the temporal location of a fiducial point, the spatial location of a fiducial point, or the amplitude of a fiducial point as discussed above. In an embodiment, a feature of a processed signal may be a calculated quantity based at least in part on a portion of the processed signal. For example, a feature of a processed signal may be a weighted or unweighted average of the processed signal over a window, a baseline value over a window, a magnitude or phase of a frequency component of a Fourier transform, a magnitude or phase or scale of a continuous wavelet transform, or any suitable calculated feature. In a further embodiment, comparing features of a first plurality of processed signals may include comparing features of portions of each of the first plurality of processed signals. For example, only the portions of a processed PPG signal corresponding to the upstroke of the received PPG signal may be used to calculate a first parameter at step 508. It has been observed that a PPG signal may change dramatically in response to a patient's breathing and posture, which may lead to false indications of changes in physiological parameters based on the PPG signal. It has also been observed that the upstroke portion of a PPG signal is relatively robust to changes during patient movement, and thus may be a more useful indicator of certain underlying physiological phenomena than other portions of a PPG signal which are more open to variation and corruption. Thus, in an embodiment, the portion of a PPG signal around the upstroke may be selectively processed and analyzed for physiological information. For example, the first set of processing operations may include a windowing operation for identifying the portions of the PPG signal that correspond to an upstroke (e.g., by analyzing a gradient or smoothed gradient of the signal to identify upstroke portions), and additional processing operations may be applied only to the upstroke portions identified by the window. In an embodiment, one or more derivatives of a PPG signal may be derived only for the portions of the PPG signal in and around an upstroke portion.

In an embodiment, only a portion or portions of the first plurality of processed signals may be analyzed to identify fiducial points or other features of interest. For example, certain segments of a signal may be identified (e.g., an upstroke portion), and only those segments may be analyzed for the presence of certain features (e.g., extrema). Identifying segments of a signal may occur before or after any one or more of the processing operations included in the first set of processing operations, and thus the segments may be identified prior to completing the processing operations. Focusing the calculation of the first parameter on identified segments of the received or processed signals may improve the efficiency of carrying out the steps of the flow diagram 500 by reducing the time spent analyzing portions of the signals that are less relevant to the information of interest (e.g., the noisier regions).

In an embodiment, calculating a first parameter at step 508 may include comparing corresponding extrema between two or more of the first plurality of processed signals. For example, a first parameter may be a time difference between the occurrence of corresponding peaks in two or more of the first plurality of processed signals. In another example, a first parameter may be a time difference between the occurrence of corresponding troughs in two or more of the first plurality of processed signals. In an embodiment, calculating a first parameter at step 508 may include comparing any corresponding fiducial points between two or more of the first plurality of processed signals. For example, a first parameter may be a time difference between the occurrence of corresponding zero crossings between two or more of the first plurality of processed signals, wherein the first set of processing operations may include taking a derivative. In another example, the first set of processing operations may include taking two time derivatives, and a fiducial point may be the minima of the first plurality of processed signals. The fiducial point may then be used to determine a first parameter.

In an embodiment, calculating a first parameter at step 508 may include comparing any fiducial points between two or more of the first plurality of processed signals, which may or may not directly correspond. For example, when the plurality of signals received at step 502 are PPG signals from a subject, a first parameter calculated at step 508 may be a time difference between a peak associated with one cardiac pulse in a first processed signal of the first plurality of processed signals, and a peak associated with a later cardiac pulse in a second processed signal of the first plurality of processed signals.

In an embodiment, the first parameter calculated at step 508 may include one or more summary statistics of similarities or differences between the first plurality of processed signals. For example, a first parameter calculated at step 508 may be an average differential pulse transit time (DPTT). Such a parameter may be calculated by averaging multiple DPTTs calculated between multiple pairs of signals from the first plurality of processed signals, based on the identification of corresponding fiducial points. Such a parameter may also be calculated by averaging multiple DPTTs calculated between a single pair or multiple pairs of signals from the first plurality of processed signals, based on the identification of multiple corresponding fiducial points (e.g., multiple corresponding peaks associated with multiple cardiac waves in a finger PPG and a forehead PPG).

In an embodiment, a first parameter may be calculated at step 508 based at least in part on a linear combination of multiple fiducial points identified in the first plurality of processed signals. For example, a weighted differential pulse transit time (DPTT_{avg}) may be calculated from one or more processed signals in accordance with:

DPTT_{avg}=x DPTT_{first}+y DPTT_{second}+(1−x−y) DPTTₚₗₑₜₕ (19)

where DPTT_{first} is a DPTT calculated between fiducial points identified in a first derivative of one or more received signals, DPTT_{second} is a DPTT calculated between fiducial points identified in a second derivative of one or more received signals, DPTTₚₗₑₜₕ is a DPTT calculated between fiducial points identified in a PPG signal which has not been differentiated (but which may have been filtered or otherwise processed), and x and y are non-negative weights whose sum is less than or equal to 1. Multiple different weighted DPTTs may be calculated and used to determine multiple different types of physiological information. For example, one weighted DPTT may be used to calculate a patient's systolic blood pressure, while another weighted DPTT may be used to calculate a patient's diastolic blood pressure. In some embodiments, different fiducials within a same set of processed signals can be used (e.g., a combination of peaks, valleys, maximum and minimum slopes identified in a first or second derivative of the signals). For example, DPTTs may be calculated from the times of the maximum peak and minimum trough of the second derivative of a pulse's upstroke, then combined via a weighted combination to provide a measurement useful in calculating mean arterial pressure (MAP).

Once a first parameter is calculated at step 508, a second parameter may be calculated at step 510 based at least in part on the second plurality of processed signals. This order of processing and calculations is merely illustrative; it will be understood that either of the first or second processing steps, and the first and second parameter calculating steps may be performed in any suitable order, or simultaneously. In an embodiment, a second parameter may be calculated at step 510 by comparing features of two or more of the second plurality of processed signals. The features that may be compared and/or the parameters that may be calculated at step 510 may take the form of any of the features and/or parameters described herein, as well as any combination of such features and/or parameters, including those described above with reference to step 508. For example, a second parameter calculated at step 508 may include a differential pulse transit time (DPTT), which may be calculated by determining the time delay between different fiducial points in the second plurality of processed signals. In an embodiment, a DPTT indicative of a patient's systolic blood pressure is determined by differentiating the PPG signals measured at two different patient body sites, identifying peaks in the differentiated PPG signals (corresponding to points of maximum positive slope in the un-differentiated PPG signals), and determining the time delay between the peaks. These PPG signals may be filtered prior to differentiating, for example, by a bandpass filter with a passband range approximately 0.5 Hz-7.5 Hz.

The first and second parameters determined at steps 508 and 510, respectively, may be determined based on processing and comparison of any number of physiological signals (e.g., multiple PPG signals), including signals in which repeating features may be identified and processed either intra- or inter-pulsewise.

At step 512, information about the subject based at least in part on the first and second parameters may be determined. In an embodiment, information determined at step 512 may be physiological information. For example, physiological information determined at step 512 may include a blood pressure of a subject (e.g., one or more of systolic and diastolic blood pressure). Some techniques that may be used to determine blood pressure based at least in part on parameters calculated from physiological signals are discussed above with reference to Eqs. 14-18. Other calculated parameters which benefit from this approach include: respiratory effort monitoring (in which changes in fiducial positioning may indicate localized changes in thoracic pressure), cardiac output monitoring (in which improvements in PPG fiducial placement and processing may benefit contour analysis techniques) and autonomic response measurements (in which heart rate variability techniques sometimes require the continuous and accurate reporting of the current pulse period).

In an embodiment, physiological information may be determined based on empirically-derived relationships between the first and second parameters and the physiological information. For example, a first parameter (e.g., an amplitude of a peak of a first derivative of a PPG signal) may be approximated by a first weighted combination of systolic blood pressure and diastolic blood pressure. Similarly, a second parameter (e.g., an amplitude of a peak of a second derivative of a PPG signal) may be approximated by a second weighted combination of systolic blood pressure and diastolic blood pressure (different from the first weighted combination). Given the first and second parameters, the systolic and diastolic blood pressures may be determined using these relationships.

After information about the subject is determined at step 512, the information determined may be output to an output device. Information may be output through a graphical representation, quantitative representation, qualitative representation, or combination of representations via output 314 (FIG. 3) and may be controlled by processor 312 (FIG. 3). In an embodiment, output 314 (FIG. 4) may transmit physiological information by any means and through any format useful for informing a patient, a care provider, or a third party, of a patient's status and may involve recording the physiological information to a storage medium. Quantitative and/or qualitative information provided by output 314 (FIG. 3) may be displayed on a display (e.g., display 28 of FIG. 2(a)). A graphical representation may be displayed in one, two, or more dimensions and may be fixed or change with time. A graphical representation may be further enhanced by changes in color, pattern, or any other visual representation. Output 314 (FIG. 3) may communicate the information by performing at least one of the following: presenting a screen on a display; presenting a message on a display; producing a tone or sound; changing a color of a display or a light source; producing a vibration; and sending an electronic message. Output 314 (FIG. 3) may perform any of these actions in a device close to a patient, or at a mobile or remote monitoring device as described previously. In an embodiment, output 314 (FIG. 3) may produce a continuous tone or beeping whose frequency changes in response to changes in a process of interest, such as a physiological process. In an embodiment, output 314 (FIG. 3) may produce a colored or flashing light that changes in response to changes in a physiological process of interest.

After or during the information determination of step 512, the steps of flow diagram 500 may be repeated. New signals may be received, or the information determination may continue on another portion of one or more of the previously received signal(s). In an embodiment, processor 312 (FIG. 3) may continuously or periodically perform steps 502-512 and update the information (e.g., as the patient's condition changes). The process may repeat indefinitely, until there is a command to stop the monitoring and/or until some detected event occurs that is designated to halt the monitoring process. For example, it may be desirable to halt a monitoring process when a detected noise has become too great, a measurement quality has become too low, or, in a patient monitoring setting, when a patient has undergone a change in condition that can no longer be sufficiently well-monitored in a current monitoring configuration. In an embodiment, processor 312 (FIG. 3) may perform the steps of flow diagram 500 at a prompt from a care provider via user inputs 56 (FIG. 2(b)). In an embodiment, processor 312 (FIG. 3) may perform the steps of flow diagram 500 at intervals that change according to patient status. For example, the steps of flow diagram 500 may be performed more often when a patient is undergoing rapid changes in physiological condition, and performed less often as the patient's condition stabilizes,

The steps of flow diagram 500 may be executed over a sliding window of a signal. For example, the steps of flow diagram 500 may involve analyzing the previous N samples of the signal, or the samples of the signal received in the previous T units of time. The length of the sliding window over which the steps of flow diagram 500 is executed may be fixed or dynamic. In an embodiment, the length of the sliding window may be based at least in part on the noise content of a signal. For example, the length of the sliding window may increase with decreasing measurement quality and/or increasing noise, as may be determined by a measurement quality assessment and/or a noise assessment. A subject's blood pressure may be monitored continuously using a moving PPG signal. PPG signal detection means may include a pulse oximeter and associated hardware, software, or both. A processor may continuously analyze the signal from the PPG signal detection means in order to continuously monitor a subject's blood pressure.

Any number of computational and/or optimization techniques may be performed in conjunction with the techniques described herein. For example, any known information regarding the physiological status of the patient may be stored in memory (e.g., ROM 52 or RAM 54 of FIG. 2(b)). Such known information may be keyed to the characteristics of the patient, which may be input via user inputs 56 (FIG. 2(b)) and used by monitor 14 (FIG. 2(b)) to, for example, query a lookup table and retrieve the appropriate information. Additionally, any of the calculations and computations described herein may be optimized for a particular hardware implementation, which may involve implementing any one or more of a pipelining protocol, a distributed algorithm, a memory management algorithm, or any suitable optimization technique.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. Accordingly, it is emphasized that the disclosure is not limited to the explicitly disclosed methods, systems and apparatuses, but is intended to include variations to and modifications thereof which are within the spirit of the following claims.

What is claimed is:

1. A method for determining physiological information about a subject, comprising:
receiving a plurality of physiological signals of a subject from one or more sensing devices; and
using one or more processing devices to:
generate a first plurality of processed signals by applying a first set of processing operations to the plurality of physiological signals;
generate a second plurality of processed signals by applying a second set of processing operations to the plurality of physiological signals, wherein the second set of processing operations is different from the first set of processing operations;
calculate a first parameter by comparing features of the first plurality of processed signals;
calculate a second parameter by comparing features of the second plurality of processed signals; and
determine physiological information about the subject based at least in part on the first and second parameters.

2. The method of claim 1, wherein the plurality of physiological signals comprises a photoplethysmograph signal.

3. The method of claim 1, wherein the plurality of physiological signals comprises two photoplethysmograph signals measured at two different body sites of the subject.

4. The method of claim 1, wherein the first set of processing operations comprises taking one or more time derivatives.

5. The method of claim 4, wherein the first set of processing operations comprises applying a low-pass filter prior to taking one or more time derivatives.

6. The method of claim 1, wherein calculating the first parameter comprises comparing extrema between one or more of the first plurality of processed signals.

7. The method of claim 1, wherein the first parameter is a differential pulse transit time.

8. The method of claim 1, wherein the physiological information comprises a blood pressure.

9. The method of claim 8, wherein the first set of processing operations comprises taking a single time derivative and the second set of processing operations comprises taking two time derivatives.

10. The method of claim 8, wherein the first parameter is determined based at least in part on a linear combination of multiple fiducial points in the first plurality of processed signals.

11. A system for determining physiological information about a subject, comprising:
a signal input configured to receive a plurality of physiological signals of a subject from one or more sensing devices; and
one or more processing devices in communication with the signal input and configured to:
generate a first plurality of processed signals by applying a first set of processing operations to the plurality of physiological signals;
generate a second plurality of processed signals by applying a second set of processing operations to the plurality of physiological signals, wherein the second set of processing operations is different from the first set of processing operations;
calculate a first parameter by analyzing features of the first plurality of processed signals;
calculate a second parameter by analyzing features of the second plurality of processed signals; and
determine physiological information about the subject based at least in part on the first and second parameters.

12. The system of claim 11, wherein the plurality of physiological signals comprises a photoplethysmograph signal.

13. The system of claim 11, wherein the plurality of physiological signals comprises two photoplethysmograph signals measured at two different body sites of the subject.

14. The system of claim 11, wherein the first set of processing operations comprises taking one or more time derivatives.

15. The system of claim 14, wherein the first set of processing operations comprises applying a low-pass filter prior to taking one or more time derivatives.

16. The system of claim 11, wherein calculating the first parameter comprises comparing extrema between one or more of the first plurality of processed signals.

17. The system of claim 11, wherein the first parameter is a differential pulse transit time.

18. The system of claim 11, wherein the physiological information comprises a blood pressure.

19. The system of claim 18, wherein the first set of processing operations comprises taking a single time derivative and the second set of processing operations comprises taking two time derivatives.

20. The system of claim 18, wherein the first parameter is determined based at least in part on a linear combination of multiple fiducial points in the first plurality of processed signals.
